(19) United States
(12) Patent Application Publication
(10) Pub. No.: US 2005/0234353 A1
(43) Pub. Date: Oct. 20, 2005

(54) Method and apparatus for analysis of non-invasive cardiac parameters

(75) Inventors: Joel Q. Xue, Germantown, WI (US); G. Ian Rowlandson, Milwaukee, WI (US); David Albert, Oklahoma City, OK (US)

Correspondence Address: MICHAEL BEST & FRIEDRICH LLP, 100 EAST WISCONSIN AVENUE, MILWAUKEE, WI, 53202-4108, US

(73) Assignee: GE Medical Systems Information Technologies, Inc., Milwaukee, WI (US)

(21) Appl. No.: 10/824,950

(22) Filed: Apr. 15, 2004

(51) Int. Cl. A61B 5/0402

(52) U.S. Cl. 600/509

(57) ABSTRACT

Method and apparatus for assessing a patient's cardiac vulnerability to sudden cardiac death. The method can include defining a relationship between depolarization and repolarization, determining a first value representative of the relationship for a first beat, determining a second value representative of the relationship for a second beat, and analyzing variation of the first value and the second value to assess the patient's cardiac vulnerability to sudden cardiac death.

### BACKGROUND OF THE INVENTION

The present invention relates to methods and apparatus for analysis of non-invasive cardiac parameters.

Sudden cardiac death generally results from disruption of a patient's heart rhythm. This disruption is often due to ventricular fibrillation. Fibrillation can occur when transient neural triggers impinge upon an electrically unstable heart causing normally organized electrical activity to become unorganized and chaotic. Much attention has been given to the to detection and prediction of cardiac vulnerability to sudden cardiac death because accurate identification of vulnerable individuals through non-invasive assessment could dramatically reduce the lives lost annually to sudden cardiac death.

Conventional analysis of non-invasive cardiac parameters for the purpose of predicting sudden cardiac death often focuses on a single aspect of a patient's electrophysiological system [e.g., repolarization (QT interval variability), depolarization (QRS duration)] or autonomous system (e.g., heart rate variability, heart rate turbulence).

### BRIEF DESCRIPTION OF THE INVENTION

One embodiment of a method of the invention can include defining a relationship between depolarization and repolarization, determining a first value representative of the relationship for a first beat of an electrocardiogram signal, determining a second value representative of the relationship for a second beat of the electrocardiogram signal, and analyzing variation between the first value and the second value to assess a patient's cardiac vulnerability to sudden cardiac death.

### BRIEF DESCRIPTION OF THE DRAWINGS

FIG. 1 is a schematic diagram illustrating a cardiac monitoring system according to the invention.

FIG. 2 illustrates an electrocardiogram signal.

FIG. 3 is a flow chart illustrating one embodiment of a method of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect.

In addition, it should be understood that embodiments of the invention include both hardware and software components or modules that, for purposes of discussion, may be illustrated and described as if the majority of the components were implemented solely in software. However, one of ordinary skill in the art, and based on a reading of this detailed description, would recognize that, in at least one embodiment, the software based aspects of the invention may be implemented in hardware. As such, it should be noted that a plurality of hardware and software based devices, as well as a plurality of different structural components may be utilized to implement the invention.

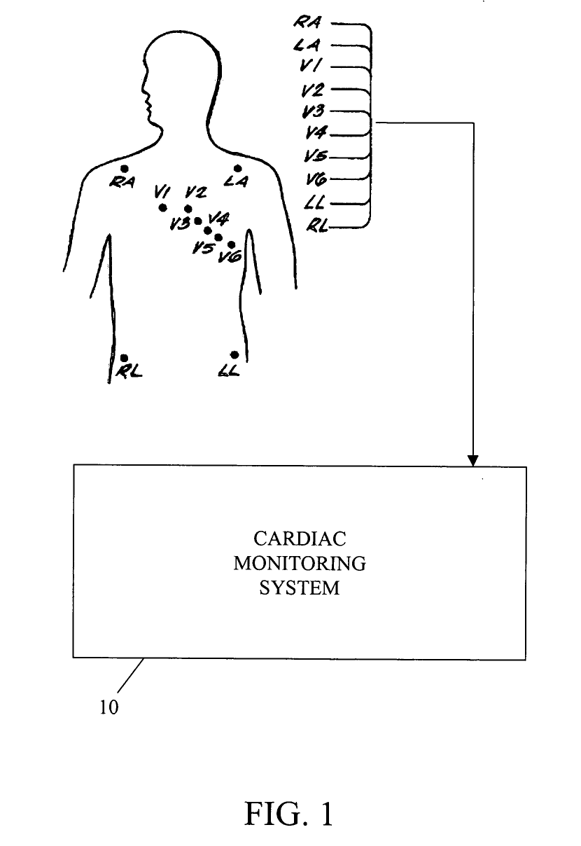

FIG. 1 illustrates a cardiac monitoring system 10 according to some embodiments of the invention. The cardiac monitoring system 10 can acquire electrocardiogram (ECG) signals, can process the acquired ECG signals to assess cardiac vulnerability to sudden cardiac death using the ECG signals, and can output data to a suitable output device (e.g., a display, a printer, and the like).

The cardiac monitoring system 10 can acquire ECG signals using an acquisition module. It should be understood that ECG data can be acquired from other sources (e.g., from storage in a memory device or a hospital information system remotely through wired or wireless networks). The acquisition module can be coupled to a patient by an array of sensors or transducers which may include, for example, electrodes coupled to the patient for obtaining an ECG signal. In the illustrated embodiment, the electrodes can include a right arm electrode RA; a left arm electrode LA; chest electrodes V1, V2, V3, V4, V5 and V6; a right leg electrode RL; and a left electrode leg LL for acquiring a standard twelve-lead, ten-electrode ECG. In other embodiments, alternative configurations of sensors or transducers (e.g., less than ten electrodes, or with extra leads to right ventricle and posterior sites as are utilized in a fifteen lead system) can be used to acquire standard or non-standard ECG signals.

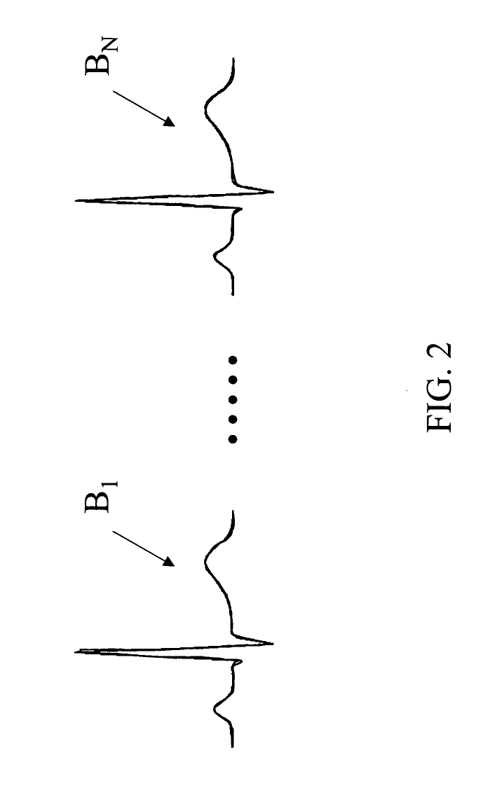

An example of an acquired ECG signal is shown in FIG. 2. The ECG signal can include N beats including beat-one B₁ through beat-N B_{N} where N is a value greater than one. The ECG signal can represent a continuous and/or a non-continuous segment of beats B₁ through B_{N}. The acquisition module can include filtering and digitization components for producing digitized ECG data representing the acquired ECG signal. In some embodiments, the ECG data can be filtered using low pass and baseline wander removal filters to remove high frequency noise and low frequency artifacts. The ECG data can, in some embodiments, be filtered by removing arrhythmic beats from the ECG data and by eliminating noisy beats from the ECG data.

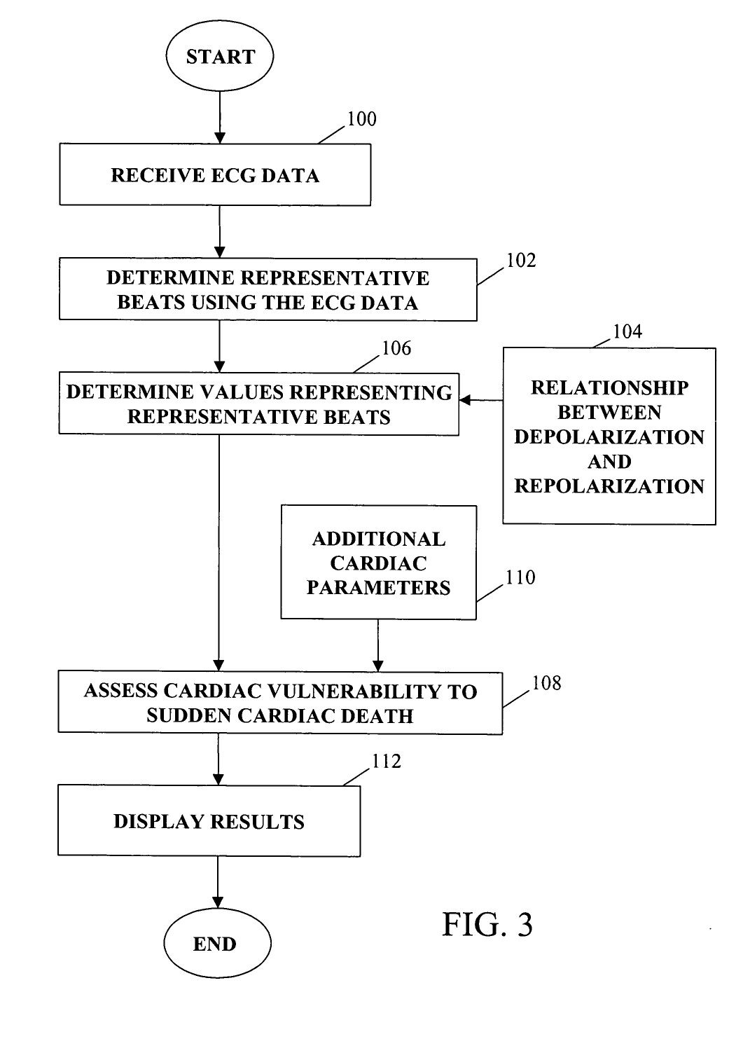

The cardiac monitoring system 10 can include a processor and memory associated with the processor. The processor can execute a software program stored in the memory to perform a method of the invention as illustrated in FIG. 3. FIG. 3 is a flow chart of a method of the invention used to assess cardiac vulnerability to sudden cardiac death using an ECG signal. Although the cardiac monitoring system 10 is described herein as including a single processor that executes a single software program, it should be understood that the system can include multiple processors, memories, and/or software programs. Further, the method of the invention illustrated in FIG. 3 can be performed manually or using other systems.

As shown in FIG. 3, the processor can receive (at 100) ECG data representing an ECG signal. The acquired ECG data can be received (e.g., from a patient in real-time via the data acquisition module, from storage in a memory device, or remotely via a network) and can be processed as necessary. The ECG data can represent continuous and/or non-continuous beats of the ECG signal. For example, in some embodiments, the ECG data can be obtained during a single time window (e.g., a ten to twenty second time window), while in other embodiments, the ECG data can be obtained during multiple time windows. Further, the ECG data can represent one or more ECG signals (e.g., a first ECG signal obtained a first day, a second ECG signal obtained a second day, and so on).

The processor can determine (at 102) representative beats using the ECG data. To facilitate determination of the representative beats, the ECG data, or a portion thereof, can be parsed into a plurality of data sets. Each data set can represent a portion of a respective beat B of the ECG signal (e.g., the QRST portion of a respective beat B of the ECG signal), a portion of a respective median beat of the ECG signal, a portion of a respective mean beat of the ECG signal, a portion of each lead of the ECG signal, and the like. The parsed data sets can be saved in an array (e.g., a waveform array). In other embodiments, the ECG data can be saved in a single data set, or alternatively, saved in multiple data sets.

As shown in FIG. 3, the processor can use at least one relationship between depolarization and repolarization 104 to determine (at 106) one or more values representing the beats. In some embodiments, the same relationship 104 is used to determine at least one value for each representative beat being analyzed. Many relationships between depolarization and repolarization 104 can be used. Several examples of relationships between depolarization and repolarization 104 are described below.

In one embodiment, the relationship between depolarization and repolarization 104 can include a QRS-T angle (e.g., a two-dimensional QRS-T angle, a three-dimensional QRS-T angle). A QRS-T angle, or QRS-T vector angle, can be determined by calculating the angular difference between a QRS vector and a T vector. There are several ways to calculate QRS and T vectors. In one embodiment, the vectors represent the maximum values of the QRS and T complexes. In another embodiment which is generally more robust, the vectors represent an average of a number of values around the maximum value. In a normal adult, the QRS-T angle is rarely greater than 60 degrees, and generally less than 45 degrees. In some embodiments, the QRS-T angle can be calculated from a set of orthoganalized X, Y, Z leads of an ECG signal, which can be either acquired or synthesized from a conventional twelve-lead ECG signal.

In another embodiment, the relationship between depolarization and repolarization 104 can include a QRS duration and a T duration. The QRS and T durations can be represented as a ratio or as another type of mathematical expression. In still another embodiment, the relationship between depolarization and repolarization 104 can include a QRS duration and a QT duration. The QRS and QT durations can be represented as a ratio or as another type of mathematical expression. In other embodiments, relationships between depolarization and repolarization 104 can include other aspects of an ECG signal which are representative of depolarization and repolarization.

The number of values determined for each representative beat using the relationship between depolarization and repolarization 104 can vary. Further, the number of representative beats analyzed using the relationship between depolarization and repolarization 104 can vary.

In some embodiments, a median or mean representative beat can be used to represent two or more beats of an ECG signal within an established time window (e.g., a 10-second to 60-second time window). A median or mean representative beat can thus provide a snap-shot of the relationship between depolarization and repolarization 104 for an established duration, rather than separately analyzing the beats.

In other embodiments, short-term trending or long-term trending can be used to represent two or more beats of an ECG signal. For short-term trending (e.g., 5 minutes to 30 minutes), two or more time windows can be analyzed relative to one another to determine variation of the relationship between depolarization and repolarization 104. The time windows can be continuous or non-continuous. Similarly, for long-term trending (e.g., 1 hour to 24 hours), two or more time windows can be analyzed relative to one another to determine variation of the relationship between depolarization and repolarization 104.

In still other embodiments, representative beats can be divided into different heart rate bins (e.g, a 30-60 beats-per-minute bin, a 60-90 beats-per-minute bin, a 90-120 beats-per-minute bin, an over 120 beats-per-minute bin, and the like). Values representative of the relationship between depolarization and repolarization 104 can then be analyzed for variation within a single bin and/or relative to other bins. In some embodiments, these variations can be compared with overall short-term trending and long-term trending values.

In some embodiments, representative beats can be obtained while a patient is being paced in an EP lab. In other embodiments, representative beats can be obtained while a patient is being paced using an implanted pacemarker. Analysis of values representative of the relationship between depolarization and repolarization 104 can then be used to determine if a paced rhythm causes changes in the variation. In still other embodiments, representative beats can be obtained while a patient is under pharmacological treatment. Analysis of values representative of the relationship between depolarization and repolarization 104 can then be used to determine if the treatment causes changes in the variation.

As shown in FIG. 3, the processor can use the values representing the relationship between depolarization and repolarization to assess (at 108) a patient's cardiac vulnerability to sudden cardiac death. In some embodiments, a single ECG analysis can be performed. In other embodiments, a time serial ECG analysis or comparison can be performed. Quantitative assessment of a patient's cardiac vulnerability to sudden cardiac death can allow for trending, which can lead to a better understanding of how heart disease affects a patient's ECG signal. This understanding can then be used to better predict sudden cardiac death and other cardiac-related diseases in all patients. Accurate techniques for non-invasive assessment of cardiac vulnerability to sudden cardiac death can allow for mass screening of individuals, with vulnerable individuals being identified for additional assessment and/or treatment.

In some embodiments, the processor can also use other cardiac parameters 110 to assess (at 108) cardiac vulnerability to sudden cardiac death. For example, other aspects of a patient's physiological system or aspects of the patient's autonomous system can be used to assess cardiac vulnerability to sudden cardiac death. Since an individual's physiology system and autonomous system are generally related, analysis of the relationship between these systems and the changes that occur in these systems over time can provide a better predictor of cardiac vulnerability to sudden cardiac death.

Heart rate variability can be used, in some embodiments, to further analyze relationships between the electrophysiology of the heart and the autonomous system. Heart rate variability can be calculated using two or more of the representative beats calculated (at 102).

Autonomous turbulence or heart rate turbulence can be used, in some embodiments, to further analyze relationships between the electrophysiology of the heart and the autonomous system. Heart rate turbulence is generally defined as the physiological, bi-phasic response of the sinus node to premature ventricular contractions (PVCs). Heart rate turbulence generally includes a short initial acceleration of the heart rate followed by a deceleration of the heart rate. Heart rate turbulence is generally quantified using a turbulence onset value and a turbulence slope value. Heart rate turbulence can be calculated using two or more of the representative beats (those calculated at 102). In one embodiment, heart rate turbulence can be analyzed for different cycle lengths of PVCs. In another embodiment, heart rate turbulence can be calculated for a time window before a PVC and a time window after the PVC. In still another embodiment, heart rate turbulence can be examined for different types of PVCs (i.e., multi-focal PVCs). In another embodiment, heart rate turbulence can be examined with a change in blood pressure. The blood pressure measurement can be obtained using continuous, non-invasive blood pressure measurements. Such analysis can allow for alignment of heart rate turbulence with different blood pressure changes and/or PVC changes, allowing for the capture of a relationship between electrophysiological change and hemodynamic change.

One method of the invention can allow patients to act as their own controls, eliminating the need for separate control groups. For example, at least one ECG signal can be obtained prior to an event (e.g., establishing a paced rhythm in the patient, delivering a pharmaceutical drug to the patient, and the like) and at least one ECG signal can be obtained during and/or after the event. The sets of ECG data can then be statistically analyzed individually and then relative to each other to determine if a statistically significant change exists. In some embodiments, normal day-to-day variability versus statistically-significant change can be measured via cluster analysis. In other embodiments, alternative statistical analyses can be used. Populations of patients can be studied and separated into separate groups based on these statistical analyses.

As shown in FIG. 3, the processor can display (at 112) the results of the various types of analyses discussed above. The results can be displayed using any suitable output device (e.g., printer, display, and the like). In some embodiments, editing tools can be used to manipulate and further analyze the results.

Various aspects of the invention are set forth in the following claims.

1. A method of assessing a patient's cardiac vulnerability to sudden cardiac death using an electrocardiogram signal, the method comprising:
defining a relationship between depolarization and repolarization;
determining a first value representative of the relationship for a first beat of the electrocardiogram signal;
determining a second value representative of the relationship for a second beat of the electrocardiogram signal; and
analyzing variation between the first value and the second value to assess the patient's cardiac vulnerability to sudden cardiac death.

2. A method as set forth in claim 1 and further comprising defining the relationship between depolarization and repolarization to include a QRS-T angle.

3. A method as set forth in claim 2 and further comprising calculating the QRS-T angle from a set of orthoganalized X, Y, and Z leads of the electrocardiogram signal.

4. A method as set forth in claim 1 and further comprising defining the relationship between depolarization and repolarization to include a QRS duration and a T duration.

5. A method as set forth in claim 1 and further comprising defining the relationship between depolarization and repolarization to include a QRS duration and a QT duration.

6. A method as set forth in claim 1 and further comprising selecting the first beat and the second beat from median beats.

7. A method as set forth in claim 1 and further comprising selecting the first beat and the second beat from mean beats.

8. A method as set forth in claim 1 and further comprising:
selecting the first beat from an electrocardiogram signal having a heart rate within a first range; and
selecting the second beat from an electrocardiogram signal having a heart rate within a second range that is different from the first range.

9. A method as set forth in claim 1 and further comprising conducting a time series analysis of the first value and the second value.

10. A method as set forth in claim 1 and further comprising using a cardiac parameter in addition to the electrocardiogram signal to assess the patient's cardiac vulnerability to sudden cardiac death.

11. A method as set forth in claim 1 and further comprising using heart rate variability in addition to the electrocardiogram signal to assess the patient's cardiac vulnerability to sudden cardiac death.

12. A method as set forth in claim 1 and further comprising using heart rate turbulence in addition to the electrocardiogram signal to assess the patient's cardiac vulnerability to sudden cardiac death.

13. A method as set forth in claim 12 and further comprising using data corresponding to blood pressure change in addition to heart rate turbulence to assess the patient's cardiac vulnerability to sudden cardiac death.

14. A method as set forth in claim 12 and further comprising calculating heart rate turbulence using a heart rate change prior to a premature ventricular contraction and a heart rate change after the premature ventricular contraction.

15. A method as set forth in claim 12 and further comprising calculating heart rate turbulence using premature ventricular contractions, the premature ventricular contractions having varying cycle lengths.

16. A method as set forth in claim 12 and further comprising calculating heart rate turbulence using premature ventricular contractions, the premature ventricular contractions having varying morphologies.

17. A method as set forth in claim 12 and further comprising:
selecting the first beat from an electrocardiogram signal obtained from the patient prior to an event; and
selecting the second beat from an electrocardiogram signal obtained from the patient at least one of during and after the event;
wherein the event includes at least one of administering a pharmaceutical drug to a patient, pacing the patient using exercise, and pacing the patient using an implanted pacemaker.

18. A method of assessing a patient's cardiac vulnerability to sudden cardiac death using an electrocardiogram signal, the method comprising:
determining a first value representative of a QRS-T angle for a first beat of the electrocardiogram signal;
determining a second value representative of a QRS-T angle for a second beat of the electrocardiogram signal; and
analyzing variation of the first value and the second value using a time series analysis in order to assess the patient's cardiac vulnerability to sudden cardiac death.

19. A method as set forth in claim 18 and further comprising using data corresponding to a cardiac parameter in addition to the electrocardiogram signal to assess the patient's cardiac vulnerability to sudden cardiac death.

20. A device for assessing a patient's cardiac vulnerability to sudden cardiac death using an electrocardiogram signal, the device comprising:
an acquisition module that acquires an electrocardiogram signal; and
an analysis module that determines a first value representative of a relationship between depolarization and repolarization for a first beat of the electrocardiogram signal, determines a second value representative of the relationship for a second beat of the electrocardiogram signal, and analyzes variation of the first value and the second value in order to assess the patient's cardiac vulnerability to sudden cardiac death.
